(19) United States
(12) Patent Application Publication
(10) Pub. No.: US 2021/0100551 A1
(43) Pub. Date: Apr. 8, 2021

(54) DUAL NEEDLE SET HAVING SINGLE OR MULTIPLE GOLD THREADS FOR HAIR LOSS TREATMENT

(71) Applicants: Jong Hwan Kim, Seoul (KR); Woo Tae Kim, Seoul (KR)

(72) Inventors: Jong Hwan Kim, Seoul (KR); Woo Tae Kim, Seoul (KR)

(21) Appl. No.: 16/071,855

(22) PCT Filed: Jan. 25, 2017
(86) PCT No.: PCT/KR2017/000856

§ 371 Date: Jul. 20, 2018

(30) Foreign Application Priority Data
Jan. 28, 2016 (KR) 10-2016-0010451

(51) Int. Cl. A61B 17/06

(52) U.S. Cl. CPC A61B 17/06066; A61B 2017/00792

(57) ABSTRACT

A dual needle set for hair loss treatment includes a thread configured with a plurality of strands and having a single or multiple gold threads, a first lone needle in which an end of the suture thread is inserted, and a second short needle in which the other end of the suture thread is inserted more easily to the same implanting hole of pre-implanted gold threads for tying or fixing. A needle part is formed at an end of the first and second needles, an installation groove is formed with a predetermined depth at the other end of the first and second needles so that the suture thread can be inserted, and the first and second needles include individually a bent part formed with a predetermined angle.

### FIELD OF THE INVENTION

The present invention relates to a dual needle set and, more particularly, to a dual needle set for a hair loss treatment in which a plurality of suture threads including a single or multiple gold threads are inserted into both ends of long and short needles.

### BACKGROUND OF THE INVENTION

It is well known that a suture thread is used as a material for a general surgical operation. The suture thread may be divided into a synthetic suture thread and a natural suture thread according to the raw materials or it may be divided into an absorbable suture thread which disappears according to a biological absorption in a certain period after suturing a treatment area and a non-absorbable suture thread which must be removed in a certain period after suturing a treatment area because the suture thread is not absorbed by a body. The non-absorbable suture thread is not dissolved and absorbed; therefore, it is used in a case that the treatment area must be maintained for a long time to achieve a treatment object.

Further, the suture thread may be divided into a monofilament suture thread formed with a single filament and a multi-filament suture thread formed by braiding a plurality of filaments according to a manufacturing method.

Differently from a suture thread used in a surgical operation, a suture thread used for plastic surgery, especially for an aging skin treatment, is sold with a needle in a state that a gold (Au) thread is fixed by attaching to an end of the needle (so called suturing needle), and a typical structure of inserting a suture thread into a suturing needle is already disclosed. A conventional connection structure between a suture thread and a suturing needle is illustrated in FIG. 1.

With reference to FIG. 1, the conventional connection structure between a suturing needle and a suture thread is established by forming an installation groove 103 having a predetermined depth in an axial direction of a suturing needle 101 at an end of the suturing needle 101 where a suture thread 104 will be attached, and by inserting an end of the suture thread 104 into the installation groove 103. After inserting the end of the suture thread 104 into the installation groove 103, the suture thread 104 is fixed to the installation groove 103 of the suturing needle 101 by filling the installation groove 103 with an adhesive material or by pressing the end of the suturing needle 101 to change the cross-section of the installation groove 103.

The connection structure of a suture thread shown in FIG. 1 may not cause any problem for inserting a single strand of suture thread into an installation groove of a suturing needle. However, in case of using a multi-filament thread formed with a plurality of fine filaments or in case of attaching a single or multiple strands of gold thread and a single strand of absorbable suture thread to a needle for an aging skin treatment, a plurality of suture threads must be inserted into the installation groove of the suturing needle. Therefore, the efficiency and reliability of adhesion can decrease because the suture threads are flexible and have a weak binding power between them.

In order to solve the above problems, “Connection structure between a suturing needle and a suture thread” has been disclosed. This connection structure enables a plurality of suture threads to be inserted into a suturing needle easily by using a connection member instead of inserting the plurality of suture threads directly into an installation groove of a suturing needle. However, blood can permeate through a crevice of the suturing needle and contaminate the suturing needle in a process of gold thread implantation because the crevice is formed in an axial direction of the suturing needle for easy insertion of the connection member into the installation groove of the suturing needle. Further, excessive bleeding can be generated in the process of implantation because a part of the connection member has a greater diameter than the outer diameter of the suture thread and the outer diameter of a partial installation groove of the suturing needle is greater than the outer diameter of the lower part of the suturing needle.

Accordingly, “Suturing needle having a suture thread” has been disclosed. This technology utilizes a method of inserting a plurality of suture threads into an installation grove of a suturing needle by pressing an end of the plurality of suture threads.

In the meantime, a needle inserted with a gold thread is currently used for an aging skin treatment; however, a problem in treatment effect and technology may occur with its use for implanting a single or multiple gold threads for a hair loss treatment. If a single gold thread is implanted, a hair growth effect is insufficient and the implanted gold thread cannot be securely fixed under a scalp. Therefore, the gold thread easily escapes and adverse effects can be markedly found in treatment effect and reliability. A treatment area of hair loss is treated with gold threads implantation by repeating a procedure of inserting and withdrawing gold threads attached needle into/from the treatment area of the hair loss. However, there is a problem in repeating the implanting process because a force must be applied to the suturing needle having a certain length in a state of gripping the suturing needle with a suturing tool.

Further, the most serious problems generated when performing a hair loss treatment by using a conventional gold thread implanting needle are: (first) that the hair growth effect is low if a single strand of gold thread is used, a longer time is required for a hair loss treatment because wounds are formed more in a larger area through repeated implanting procedures and a treatment period is extended, and a treatment effect is decreased because the inserted gold thread has no fixing or tying part and escapes easily; (second) that a longer operation time is required when inserting a large amount of gold threads because only the single strand of thread is implanted at every insertion; and (third) that inflammation can be generated more easily because of separation or displacement of the implanted gold thread.

In order to solve the above problems, the implanted gold threads must be fixed or tied to avoid separation or displacement, but it may move slightly to stimulate a scalp and then must avoid inflammation. Otherwise, a satisfactory effect cannot be obtained in hair loss treatment with gold thread implantation.

Further, in case that a tie must be formed through the same implanting hole of a needle in an implanting process, there is a problem that a separate needle must be prepared.

### SUMMARY OF THE INVENTION

In order to solve the above problems, an object of the present invention is to provide a dual needle set in which a plurality of suture thread strands including a single or multiple gold threads are inserted into needles having different lengths from each other.

Another object of the present invention is to provide a dual needle set enabling an easy treatment by bending a plurality of needles having different lengths in a predetermined angle, and the plurality of needles is connected to both ends of a suture thread.

A further other object of the present invention is to provide a dual needle set having different needle lengths capable of a stable attachment by forming a coating membrane at both ends of a suture thread and inserting the suture thread formed with the coating membrane into an installation groove of a suturing needle.

A further other object of the present invention is to provide a dual needle set which can improve a treatment effect by increasing an amount of gold thread inserted into a hair loss area and avoid displacement of the gold thread even though the gold thread is simply inserted and buried in an aging skin treatment.

In order to achieve the above object, a dual needle set for a hair loss treatment according to an embodiment of the present invention may include a suture thread configured with a plurality of strands and having a single or multiple gold threads, a first needle having a long length in which an end of the suture thread is inserted, and a second needle having a short length in which the other end of the suture thread is inserted to the same implanting hole to make tying or fixing more easily. A needle part is formed at an end of the first and second needles, an installation groove is formed with a predetermined depth at the other end of the first and second needles so that the suture thread can be inserted, and the first and second needles have different lengths from each other and include individually a bent part formed with a predetermined angle.

The present invention provides the following effects through the above technical solutions.

According to various embodiments of the present invention, because suture threads have a structure of enabling pressing of both ends and a coating membrane, the suture threads can be easily inserted into an installation groove of the needle and attached stably, even though a crevice is not formed in the installation groove. Therefore, excessive bleeding generated in a process of implanting a gold thread in a skin can be suppressed because a needle having a predetermined outer diameter is provided and permeation of the blood through the crevice of the needle and contamination of the needle can be avoided in the process of gold thread implantation procedure.

Further, according to various embodiments of the present invention, a treatment time can be reduced markedly because both ends of a suture thread are inserted into needles having different lengths and bent in a predetermined angle and a plurality of gold threads is installed in a dual needle set so that a single or multiple gold threads having a plurality of strands or a thicker diameter can be inserted in an implantation treatment. An effect on hair growth is markedly improved because the inserted gold thread can be fixed or tied to avoid displacement of the gold thread and wounds or inflammation can be reduced markedly compared with the conventional method.

Further, according to various embodiments of the present invention, the treatment effect is further improved because each gold thread can be fixed or tied easily at the same location of needle hole by forming a knot through long and short needles so that displacement of gold threads is prevented after inserting the gold threads having a plurality of strands or a thick diameter at the same time, even though both ends of the gold threads are located at the same location.

### BRIEF DESCRIPTION OF THE DRAWINGS

FIG. 1 is a cross-sectional view illustrating a conventional needle and a conventional connection structure of a suture thread.

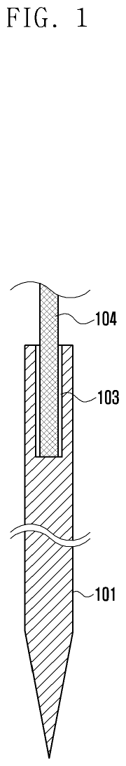

FIG. 2 is an exploded perspective view illustrating a dual needle set according to an embodiment of the present invention.

FIG. 3 is a perspective view illustrating a connection state between a first needle and a suture thread of FIG. 2.

FIG. 4 is a cross-sectional view illustrating a cross section cut along the line I-I′ of FIG. 3.

### DETAILED DESCRIPTION

Hereinafter, embodiments of the present invention are described in detail with reference to the accompanying drawings. The same reference symbols are used throughout the drawings to refer to the same or like parts. Detailed descriptions of well-known functions and structures incorporated herein may be omitted to avoid obscuring the subject matter of the present invention.

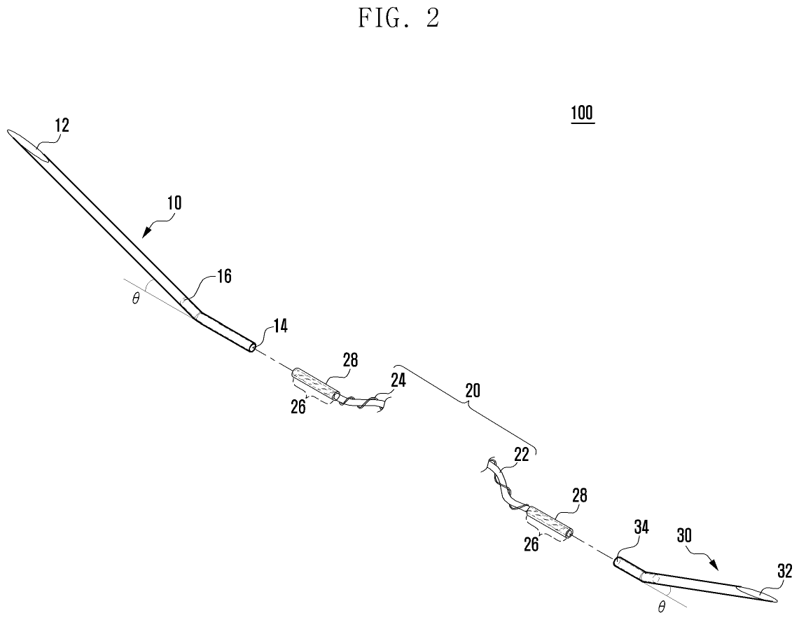

Hereinafter, an embodiment of the present invention is described with reference to FIGS. 2 to 4.

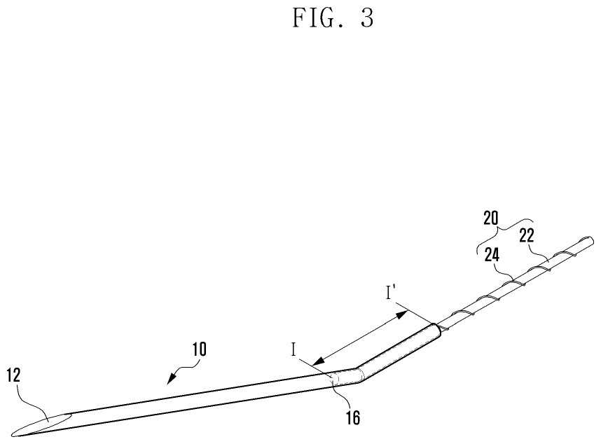

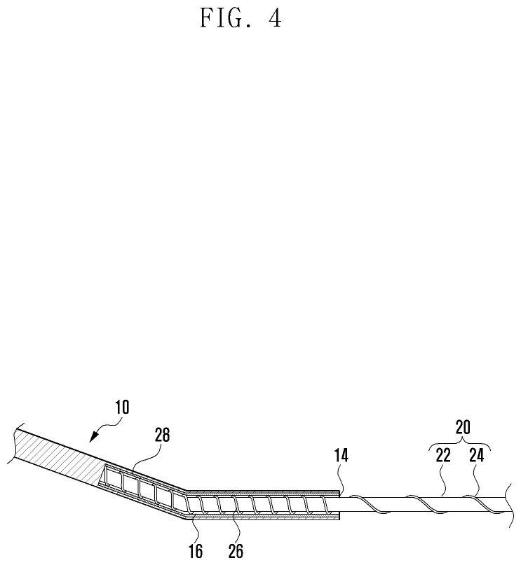

FIG. 2 is an exploded perspective view illustrating a dual needle set according to an embodiment of the present invention, FIG. 3 is a perspective view illustrating a connection state between a first needle and a suture thread of FIG. 2, and FIG. 4 is a cross-sectional view illustrating a cross section cut along the line I-I′ of FIG. 3.

As shown in FIGS. 2 to 4, the dual needle set 100 according to an embodiment of the present invention may include a first needle 10 into which an end of a suture thread 20 is inserted and a second needle 30 into which the other end of the suture thread 20 is inserted.

Needle tips 12 and 32 being inserted into a biological tissue are formed at ends respectively of the first needle 10 and the second needle 30, and installation grooves 14 and 34 having a predetermined depth are formed in an axial direction of the needle so that the end of the suture thread 20 can be inserted into the other ends of the needles.

The first needle 10 is a long needle having a greater length than the second needle 30, and the second needle 30 is a short needle having a smaller length than the first needle 10. Preferably, the length of the second needle 30 may be ½ to ⅓ of the length of the first needle 30.

In the meantime, the first needle 10 and the second needle 30 may include bent parts 16 and 36 formed with a predetermined angle. The bent parts 16 and 36 may be formed with an angle θ in the range of 10 to 20 degrees, and they may be located adjacent to inner sides of the installation grooves 14 and 34 respectively of the first needle 10 and the second needle 30.

The suture thread 20 installed and fixed in the installation grooves 14 and 34 of the first and second needles 10 and 30 may include an absorbable suture thread 22 and a non-absorbable suture thread 24 wound on the surface of the absorbable suture thread 22 at predetermined intervals.

The diameter of the absorbable suture thread 22 may be greater than the diameter of the non-absorbable suture thread 24. For example, in case that hairs exist in a treatment area, the non-absorbable suture thread 24 can be configured with very fine gold thread having a diameter of 0.1 mm to promote hair formation and growth of biological tissues and, in some cases, a thicker gold thread or a plurality of fine gold threads can be used.

An end of the suture thread 20 may include a compressed part 26 so that the suture thread can be easily inserted into the installation grooves 14 and 34 of the first and second needles 10 and 30. Namely, if the end of the suture thread 20 is compressed by using certain heat and pressure, the non-absorbable suture thread 24 having a smaller diameter adheres to the absorbable suture thread 22 having a greater diameter, and thereby the suture threads can be handled like a single thread because the suture threads do not move independently.

Further, a coating membrane 28 can be included in the compressed part 26. By forming the coating membrane 28 in the compressed part 26, the suture thread 20 can be easily inserted into the installation grooves 14 and 34 of the first and second needles 10 and 30, the suture thread 20 inserted into the installation grooves 14 and 34 can be fixed by applying pressure with a tweezers, and a tie-down stability of a suture thread can be improved.

The coating membrane 28 can be formed by using silicon. Further, because a conventional connection member is not required, the outer diameter of the compressed part 26 of the suture thread 20 can be prevented from increasing, and dual needles 10 and 30 having the same diameter can be formed. Accordingly, the outer diameters of the first and second needles 10 and 30 formed with the installation grooves 14 and 34 can be prevented from increasing, and excessive bleeding generated by a diameter difference between the needles 10 and 30 can be suppressed in a process of gold thread implantation. Further, because a crevice does not have to be formed in an axial direction of the needles 10 and 30, permeation of the blood through the crevice and contamination of the needle can be avoided in the process of gold thread implantation.

In the embodiment of the present invention, an example has been described for inserting and installing ends of 2 suture thread strands (an absorbable suture thread and a single or multiple gold threads as a non-absorbable suture thread) into the first needle 10 and the second needle 30; however, the diameter of the non-absorbable suture thread (0.1 mm gold thread) can be increased to 0.2-0.3 mm, a plurality of gold thread strands can be installed, or non-absorbable suture threads of gold thread having different diameters can be used. Even when using non-absorbable suture threads of gold thread having different diameters, a compressed part 26 and a coating membrane 28 are formed in the suture thread 20, and the outer diameters of the compressed part 26 and the coating membrane 28 of the suture thread 20 are preferably formed less than the inner diameter of the installation grooves 14 and 34 of the needles 10 and 30 respectively so that a formed compressed part 26 and coating membrane 28 can be stably inserted into the installation grooves 14 and 34 of the needles 10 and 30 respectively.

Further, the compressed parts 26 of the suture thread 20 are preferably formed longer than the length of the installation groove 14 of the long needle 10, inserted into the inner part of the installation grooves 14 and 34, and finally inserted into the bent parts 16 and 36 formed within an angle in the range of 10 to 20 degrees. Accordingly, the suture thread 20 can be more securely fixed in the installation grooves 14 and 34.

The dual needle set according to the present invention implants a single or multiple gold threads in a treatment area of hair loss by repeating a procedure of inserting and withdrawing the first needle (long needle) 10 having a greater length than the second needle 30 into/from the biological tissue of the treatment area including hair loss. Subsequently, the second needle (short needle) 30 is withdrawn through a hole formed by passing the first needle 10, and the treatment is completed by forming a knot and inserting the knot into the hole.

According to another embodiment of the present invention, various types of gold thread can be implanted into a treatment area of hair loss by using various methods, and then displacement of the implanted gold thread can be prevented. For example, when implanting a single or multiple gold threads into a treatment area in a round form, the treatment can be completed by forming a knot as described above, or when implanting a gold thread into a treatment area in a grid form, the treatment can be completed by anchoring at the same implanting hole with gold threads of long needle and other gold threads of short needle, which can be inserted more easily to the same needle hole of pre-implanted longer needle.

As described above, a dual needle set according to the present invention can promote formation and growth of hairs by using a suture thread configured with a non-absorbable gold thread having a great diameter or with a plurality of gold threads, and a surgical treatment can be easily performed by using long and short needles inserted with a suture thread in a bent form at both ends.

The above description relates to an example of preferred embodiments of the present invention, and thereby the present invention is not limited to the above embodiments. For example, it has been described that a needle is formed by bending in the embodiments; however, a curved needle can be applied also if necessary.

Although embodiments of the disclosure have been described in detail hereinabove, it should be understood that many variations and modifications of the basic inventive concept described herein will still fall within the spirit and scope of the disclosure as defined in the appended claims.

1. A dual needle set for a hair loss treatment, the dual needle set comprising:
a suture thread configured with a plurality of strands and having a single or multiple gold threads;
a first needle in which an end of the suture thread is inserted; and
a second needle in which the other end of the suture thread is inserted,
wherein a needle part is formed at an end of the first and second needles, an installation groove is formed with a predetermined depth at the other end of the first and second needles so that the suture thread can be inserted, and the first and second needles have different lengths from each other and include individually a bent part formed with a predetermined angle.

2. The dual needle set of claim 1, wherein the first needle is a long needle having a greater length than the second needle, and the second needle is a short needle having a smaller length than the first needle.

3. The dual needle set of claim 1, wherein the bent part is located adjacent to an inner side of each installation groove of the first and second needles.

4. The dual needle set of claim 1, wherein the suture thread includes a strand of absorbable suture thread and a plurality of non-absorbable suture threads, the non-absorbable suture thread being a gold thread which is formed with a plurality of strands, great diameter, or by twisting a plurality of strands.

5. The dual needle set of claim 1, wherein a compressed part is formed at an end of the suture thread.

6. The dual needle set of claim 1, wherein a coating membrane is formed at an end of the suture thread.

7. The dual needle set of claim 5, wherein a bent part is formed at the needle, and the compressed parts or coating membranes of the suture thread are inserted into the installation groove of the needle through the bent part.

8. The dual needle set of claim 6, wherein a bent part is formed at the needle, and the compressed parts or coating membranes of the suture thread are inserted into the installation groove of the needle through the bent part.
